(19) United States
(12) Patent Application Publication
(10) Pub. No.: US 2014/0187796 A1
(43) Pub. Date: Jul. 3, 2014

(54) PROCESS FOR THE TREATMENT OF A RECYCLYING STREAM FROM A PLANT FOR THE PRODUCTION OF POLYARYLENE ETHER SULFONES

(71) Applicant: BASF SE, Ludwigshafen (DE)

(72) Inventors: Christoph Sigwart, Jeollanam-do (KR); Jutta Vonend, Bad Dürkheim (DE); Cornelis Hendricus De Ruiter, Mannheim (DE); Jörg Erbes, Karlsruhe (DE)

(73) Assignee: BASF SE, Ludwigshafen (DE)

(21) Appl. No.: 14/140,642

(22) Filed: Dec. 26, 2013

Related U.S. Application Data
(60) Provisional application No. 61/746,575, filed on Dec. 28, 2012.

(51) Int. Cl. C07D 207/267

(52) U.S. Cl. CPC C07D 207/267
USPC 548/555

(57) ABSTRACT

A process is proposed for the treatment of a recycling stream (1) from a plant for the production of polyarylene ether sulfones via polycondensation of aromatic bishalogen compounds and of aromatic bisphenols or their salts in the presence of at least one alkali metal carbonate or ammonium carbonate or alkali metal hydrogencarbonate or ammonium hydrogencarbonate in an N-alkyl-2-pyrrolidone as solvent, comprising
from 60 to 90% by weight of water,
from 10 to 40% by weight of N-alkyl-2-pyrrolidone and, as contaminant detrimental to specification, up to 5000 ppm by weight of the alkylsuccinimide corresponding to the N-alkyl-2-pyrrolidone and, alongside this, up to 1000 ppm by weight of other substances with higher boiling point than N-alkyl-2-pyrrolidone, in particular inorganic salts, based in each case on the total weight of the recycling stream (1), where the entirety of the components gives 100% by weight,
   giving a pure N-alkyl-2-pyrrolidone stream (2) which can be returned to the plant for the production of polyarylene ether sulfones, via a final distillation in a final column (K), which comprises preceding the final distillation by a preliminary purification by evaporation in one or more evaporator stages for reducing the level of inorganic salts, where one or more vapor streams (3, 4, 5) are obtained, which are introduced as feed streams into the final column (K), and where the bottom stream from the last evaporator stage is removed, and where a substream (9) comprising 1.0% by weight to 10% by weight of the bottom stream (7) from the final column (K) is removed, and the rest of the bottom stream from the final column (K) is recycled into the last evaporator stage.

### CROSS-REFERENCE TO RELATED APPLICATIONS

This application claims the benefit of U.S. Provisional Application 61/746,575, filed Dec. 28, 2012, which is incorporated herein by reference.

The invention relates to a process for the treatment of a recycling stream from a plant for the production of polyarylene ether sulfones via polycondensation of aromatic bishalogen compounds and of aromatic bisphenols or their salts in the presence of at least one alkali metal carbonate or ammonium carbonate or alkali metal hydrogencarbonate or ammonium hydrogencarbonate in an N-alkyl-2-pyrrolidone as solvent.

Polyarylene ether sulfones are known with trademark Ultrason® from BASF SE and comprise in particular polyether sulfones (Ultrason® E), polysulfones (Ultrason® S) and polyphenyl sulfones (Ultrason® P).

Ultrason® E, Ultrason® S, and Ultrason® P are transparent plastics with high temperature resistance. They are used in many applications in engineering and in the electrical/electronics sector. There are also numerous reasons for a use as replacement for glass, metal, ceramic, and porcelain in the food-and-drinks sector and household sector: heat resistance extending to 180° C. or short periods at 220° C., good mechanical properties and high breakage resistance, resistance to superheated steam, and excellent resistance to chemicals.

Ultrason® E, S, and P are amorphous thermoplastic polymers with the following underlying structure:

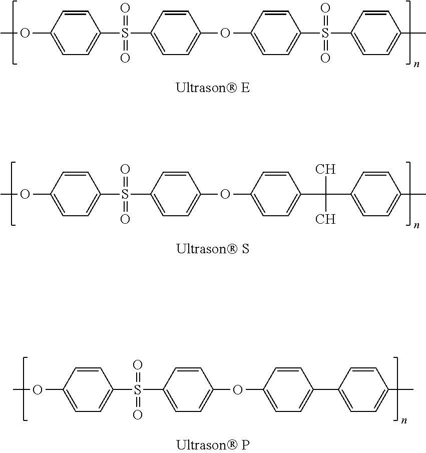

Moldings made of Ultrason® not only have high dimensional stability but also strength, stiffness, and toughness, these properties extending to the vicinity of the glass transition temperature.

The most important features of Ultrason® are:
properties independent of temperature
very high long-term service temperatures
good dimensional stability
high stiffness
high mechanical strength
good electrical insulation capability
advantageous dielectric properties
very advantageous fire performance
exceptional resistance to hydrolysis

The three Ultrason® parent polymers are amorphous thermoplastics and are transparent. However, by virtue of the high temperatures required during their production and processing they have a certain intrinsic color (pale golden yellow to ocher) which prevents achievement of the theoretically possible transmittance values for visible light. The qualities achievable currently are nevertheless suitable for very many transparent applications. Ultrason® also has high refractive indices in the visible wavelength region, and it therefore has another use in functional optical applications, for example lenses for electronic cameras.

Polyarylene ether sulfones are frequently produced via polycondensation in the presence of, as polar aprotic solvent, an N-alkyl-2-pyrrolidone, hereinafter abbreviated to NAP. N-methyl- or N-ethylpyrrolidone are particular N-alkyl-2-pyrrolidones used, and preferably N-methylpyrrolidone is used. Processes of this type are disclosed by way of example in U.S. Pat. No. 4,870,153, EP-A 113 112, EP-A 297 363, and EP-A 135 130.

Contaminated solvent arises in the above processes, and for economic and environmental reasons has to be treated and recycled into the process.

However, the solvent used in the above processes has to comply with the criteria for what is known as pure NAP, i.e. at least 99.0% by weight NAP content or else at least 99.5% by weight NAP content, or else at least 99.8% by weight NAP content, based in each case on the total weight of the pure NAP stream, and at most the following contents of components detrimental to specification: 0.1% by weight of water and 0.01% by weight of N-alkylsuccinimide, hereinafter abbreviated to NAS, based in each case on the total weight of the pure NAP stream.

Higher NAS contents in the NAP solvent have a disadvantageous effect on the color of the polyarylene ether sulfone, which is the useful product. This is surprising because not only NAP itself but also NAS, which can be produced by way of example via oxidation of NAP by atmospheric oxygen, are colorless substances. However, for the reasons described the market demands polyaryl ether sulfones with minimized intrinsic color.

Current thinking in relation to polyarylene ether sulfone production with NAP as solvent is that there is a causal connection between the NAS produced via oxidation of the NAP, for example the N-methylsuccinimide (NMS) produced via oxidation of N-methylpyrrolidone (NMP):

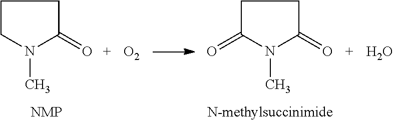

and the undesired intrinsic color of the final polyarylene ether sulfone product.

It is believed that NAS is a precursor for higher-molecular-weight colorant components which impair the intrinsic color of the final polyarylene ether sulfone product.

Before NAP-containing recycling streams are recycled into the production of polyarylene ether sulfone, they are therefore purified by final distillation in a traditional distillation column sufficiently to give a pure NAP complying with the criteria defined above.

CN 2007 100 39497 discloses a process for the reclamation of NMP as solvent from the polycondensation process to give para-phenyleneterephthalamide, where the polymer is washed with deionized water, the wash solution is neutralized with a carbonate, oxide or hydroxide of an alkali metal or of an alkaline earth metal, and in two thin-layer evaporators, at a pressure of from 0.1 to 3.0 bar absolute and at a temperature of from 90 to 120° C. is subjected to initial distillation, and also then to final distillation, giving a pure NMP stream with purity higher than 99.5% and with water content below 100 ppm which is suitable for return into the polycondensation plant for the production of polymerizable para-phenyleneterephthalamides.

When a conventional procedure, without preliminary evaporation, is used the heat exchanger for the bottom stream from the distillation column for pure NAP becomes blocked by contaminants after only a short time, and said plant therefore requires frequent shutdown for heat exchanger cleaning.

### BRIEF DESCRIPTION OF THE DRAWING

FIG. 1 shows a diagram of a preferred plant for carrying out the process of the invention.

### DETAILED DESCRIPTION OF THE INVENTION

In the light of this, it was an object of the invention to provide a process for the treatment of recycling streams from polyarylene ether sulfone processes via distillation to give pure NAP which can be recycled into the plant for carrying out a polyarylene ether sulfone process, where the process reliably provides an increased operation time of the distillation column and moreover minimizes required apparatus cost and energy cost, and where NAP losses are minimized. In particular, through additional removal of high boilers from the final column, the intention is to allow greater concentration of N-alkylsuccinimide in the bottom and hence, in turn, to achieve minimization of the bottom removal volume and reduction of the NAP losses.

The object is achieved via a process for the treatment of a recycling stream from a plant for the production of polyarylene ether sulfones via polycondensation of aromatic bishalogen compounds and of aromatic bisphenols or their salts in the presence of at least one alkali metal carbonate or ammonium carbonate or alkali metal hydrogencarbonate or ammonium hydrogencarbonate in N-alkyl-2-pyrrolidone as solvent, comprising
from 60 to 90% by weight of water,
from 10 to 40% by weight of N-alkyl-2-pyrrolidone and, as contaminant detrimental to specification, up to 5000 ppm by weight of the alkylsuccinimide corresponding to the N-alkyl-2-pyrrolidone and, alongside this, up to 1000 ppm by weight of other substances with higher boiling point than N-alkyl-2-pyrrolidone, in particular inorganic salts, based in each case on the total weight of the recycling stream, where the entirety of the components gives 100% by weight,
   giving a pure N-alkyl-2-pyrrolidone stream which can be returned to the plant for the production of polyarylene ether sulfones, via a final distillation process in a final column, which comprises preceding the final distillation by a preliminary purification by evaporation in one or more evaporator stages for reducing the level of inorganic salts, where one or more vapor streams are obtained, which are introduced as feed streams into the final column, and where the salt-containing bottom stream from the last evaporator stage is removed, and where a substream comprising 1.0 to 10% by weight of the bottom stream from the final column is removed, and the rest of the bottom stream from the final column is recycled into the last evaporator stage.

It has been found to be possible to treat recycling streams from the production of polyarylene ether sulfones in a manner which is advantageous in terms of apparatus and of energy to give pure NMP, by preliminary purification via evaporation being carried out upstream of the final distillation in a conventional distillation column, in which preliminary purification, in one or more evaporator stages, the content of salts of the recycling stream is reduced.

The recycling stream preferably comprises from 70 to 85% by weight of water, from 25 to 30% by weight of N-alkyl-2-pyrrolidone and, as contaminant detrimental to specification, up to 1000 ppm by weight of the alkylsuccinimide corresponding to the N-alkyl-2-pyrrolidone and, alongside this, up to 300 ppm by weight of other substances with higher boiling point than N-methylpyrrolidone, in particular inorganic salts, based in each case on the total weight of the recycling stream, where the entirety of the components gives 100% by weight.

A process is preferred wherein the N-alkyl-2-pyrrolidone is N-ethylpyrrolidone or N-methylpyrrolidone, preferably N-methylpyrrolidone.

Preferably two, more preferably three, evaporator stages are provided for the evaporation.

The first evaporator stage is preferably operated with a pressure in the vapor space in the range from 250 mbar absolute to atmospheric pressure, in such a way that most, in particular from 70 to 90%, of the water comprised in the recycling stream is drawn off by way of the vapor stream from the first evaporator stage which is introduced as feed stream to the final column.

More preferably the first evaporator stage is operated at a pressure in the vapor space in the range from 300 to 800 mbar absolute.

The second evaporator stage is preferably operated at a pressure in the vapor space in the range from 250 to 500 mbar absolute, in such a way that most, in particular from 90 95%, of the N-alkyl-2-pyrrolidone comprised in the recycling stream, in particular N-methylpyrrolidone, is drawn off by way of the vapor stream from the second evaporator stage, this stream being introduced as feed stream to the final column.

The second evaporator stage is advantageously operated at a pressure in the vapor space in the range from 300 to 400 mbar.

The third evaporator stage is preferably operated at a pressure in the vapor space in the range from 100 to 400 mbar.

In a preferred embodiment, a third evaporator stage is provided. The third evaporator stage is advantageously operated at a pressure in the vapor space in the range from 100 to 200 mbar.

It is particularly preferable to use a thin-layer evaporator as evaporator in the third evaporation stage. This is less susceptible to crusting by deposits.

The vapor stream from the second evaporator stage is advantageously introduced into the final column above the vapor stream from the third evaporator stage and the vapor stream from the first evaporator stage is introduced into the final column above the vapor stream from the second evaporator stage.

The final column is preferably designed having 15 to 35, in particular 20 to 30, theoretical separation stage.

The final column is preferably operated at an overhead pressure at which it is still possible to use river water for cooling at the top of the column, in particular at an overhead pressure in the range from 150 to 250 mbar absolute, more preferably at about 200 mbar absolute. The bottom temperature in the final column is preferably adjusted to about 160 to 170° C., so that the bottom stream still comprises about 0.5 to 10% by weight of NMS, in particular still comprises about 1.0 to 5% by weight of NMS.

The invention is explained in more detail below with reference to a drawing, and also to an inventive example:

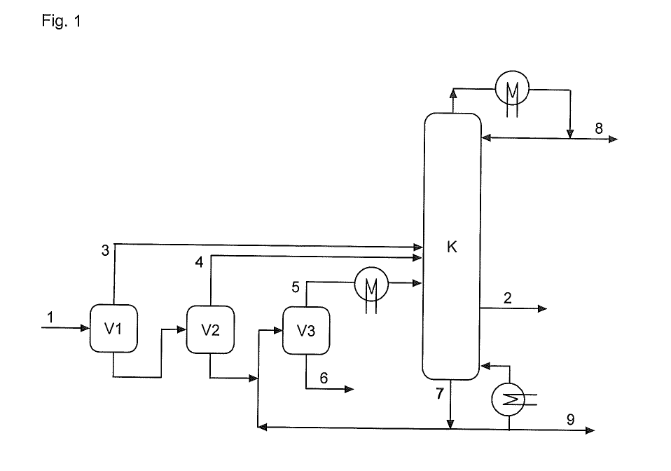

The single FIGURE, FIG. 1, is a diagram of a preferred plant for carrying out the process.

A NMP-containing recycling stream 1 is introduced into the first evaporator stage V1, from which a vapor stream 3 predominantly comprising water is drawn off and introduced as feed stream into final column K. The bottom stream from the first evaporator stage V1 is introduced into the second evaporator stage V2; from this a further vapor stream 4 is drawn off and introduced as further feed stream into the final column K.

The bottom stream from the second evaporator stage V2 is introduced into the third evaporator stage V3. From this, a further vapor stream 5 is drawn off, condensed and is introduced, as liquid feed stream, into the final column K.

A salt-containing bottom stream 6 is discharged from the third evaporator stage V3. The following are drawn off from the final column K: a pure NMP stream 2 from the stripping section thereof, preferably in a gaseous form, as side stream, a bottom stream 7, which is partially discharged as stream 9 and furthermore recycled into the third evaporator stage V3, and also an overhead stream 8 which predominantly comprises water and which is sent for disposal.

### INVENTIVE EXAMPLE

The Aspen® simulation program from Aspen Technology Inc. was used to simulate a process for the treatment of a recycling stream 1 for a plant corresponding to the diagram in FIG. 1, whereupon the values listed in the table below were obtained for the composition of the streams.

The following operating conditions were assumed:

For the evaporation in the first evaporator stage V1 a pressure of 350 mbar absolute and a temperature of 80° C., for the second evaporator stage V2 likewise a pressure of 350 mbar absolute and a temperature of 128° C., for the third evaporator stage V3 a pressure of 150 mbar absolute and a temperature of 137° C., and for the final column K an overhead pressure of 197 mbar absolute and a temperature of 60° C. at the top of the column, or else a pressure of 337 mbar absolute and a bottom temperature of 163° C.

As can be seen from the table, NMP loss across the entire process is 1.21% (based on NMP introduced into the process by way of the recycling stream 1). NMS content in the pure NMP stream is 84 ppm by weight.

| | | | Pure NMP | | Overhead | | Bottom stream | | Stream 9 | |
|---|---|---|---|---|---|---|---|---|---|---|
| | | | stream 2 (side | | stream 8 | | 6 from third | | discharged | |
| | Recycling | | outlet) from final | | from final | | evaporator | | from bottom of | |
| | stream 1 | | column K | | column K | | stage V3 | | final column K | |
| (column) | kg/h | % | kg/h | % | kg/h | % | kg/h | % | kg/h | % |
| H₂O | 739.9 | 74.0 | 0.0 | 0.0 | 739.6 | 100 | 0.001 | 0.1 | 0.0 | 0.0 |
| KCl | 0.012 | 0.001 | 0.0 | 0.0 | 0.0 | 0.0 | 0.012 | 1.2 | 0.0 | 0.0 |
| NMP | 260.0 | 26.0 | 256.9 | 100.0 | 0.0 | 0.0 | 1.0 | 97.2 | 2.1 | 97.3 |
| NMS | 0.100 | 0.01 | 0.022 | 0.0084 | 0.0 | 0.0 | 0.017 | 1.6 | 0.059 | 2.75 |
| Total | 1000 | 100.0 | 256.9 | 100.0 | 739.6 | 100.0 | 1.1 | 100.0 | 2.2 | 100 |

| | |
|---|---|
| NMP | 1.21% |
| loss | |

1-12. (canceled)

13. A process for the treatment of a recycling stream from a plant for the production of polyarylene ether sulfones via polycondensation of aromatic bishalogen compounds and of aromatic bisphenols or their salts in the presence of at least one alkali metal carbonate or ammonium carbonate or alkali metal hydrogencarbonate or ammonium hydrogencarbonate in an N-alkyl-2-pyrrolidone as solvent, comprising
from 60 to 90% by weight of water,
from 10 to 40% by weight of the N-alkyl-2-pyrrolidone and, as contaminant detrimental to specification, up to 5000 ppm by weight of the alkylsuccinimide corresponding to the N-alkyl-2-pyrrolidone and, alongside this, up to 1000 ppm by weight of other substances with higher boiling point than N-alkyl-2-pyrrolidone, in particular inorganic salts, based in each case on the total weight of the recycling stream, where the entirety of the components does not exceed 100% by weight,
giving a pure N-alkyl-2-pyrrolidone stream which can be returned to the plant for the production of polyarylene ether sulfones, via a final distillation in a final column which comprises preceding the final distillation by a preliminary purification by evaporation in one or more evaporator stages for reducing the level of inorganic salts, where one or more vapor streams are obtained, which are introduced as feed streams into the final column, and where the bottom stream from the last evaporator stage is removed, and where a substream comprising 1.0% by weight to 10% by weight of the bottom stream from the final column is removed, and the rest of the bottom stream from the final column is recycled into the last evaporator stage.

14. The process according to claim 13, wherein the recycling stream (1) comprises
from 70 to 85% by weight of water,
from 25 to 30% by weight of N-alkyl-2-pyrrolidone and, as contaminant detrimental to specification, up to 1000 ppm by weight of the alkylsuccinimide corresponding to the N-alkyl-2-pyrrolidone and, alongside this, up to 300 ppm by weight of other substances with higher boiling point than N-methylpyrrolidone, based in each case on the total weight of the recycling stream,
where the entirety of the components does not exceed 100% by weight.

15. The process according to claim 13, wherein the N-alkyl-2-pyrrolidone is N-ethyl-pyrrolidone or N-methylpyrrolidone.

16. The process according to claim 13, wherein two or three evaporator stages are provided.

17. The process according to claim 16, wherein the first evaporator stage is operated at a pressure in the vapor space in the range from 250 mbar absolute to atmospheric pressure, such that from 70% to 90% of the water present in the recycling stream is drawn off by way of the vapor stream from the first evaporator stage, said stream being introduced as feed stream into the final column.

18. The process according to claim 17, wherein the first evaporator stage is operated at a pressure in the vapor space in the range from 300 to 800 mbar absolute.

19. The process according to claim 16, wherein the second evaporator stage is operated at a pressure in the vapor space in the range from 250 to 500 mbar absolute, such that from 90% to 95%, of the N-alkyl-2-pyrrolidone present in the recycling stream is drawn off by way of the vapor stream from the second evaporator stage, said stream being introduced as feed stream into the final column.

20. The process according to claim 19, wherein the second evaporator stage is operated at a pressure in the vapor space in the range from 300 to 400 mbar absolute.

21. The process according to claim 13, wherein the third evaporator stage is operated at a pressure in the vapor space in the range from 100 to 400 mbar absolute.

22. The process according to claim 21, wherein the third evaporator stage is operated at a pressure in the vapor space in the range from 100 to 200 mbar absolute.

23. The process according to claim 13, wherein a thin-film evaporator is used as evaporator in the third evaporator stage.

24. The process according to claim 13, wherein the vapor stream from the second evaporator stage of the final column is introduced above the vapor stream from the third evaporator stage, and the vapor stream from the first evaporator stage of the final column is introduced above the vapor stream from the second evaporator stage.

25. The process according to claim 15, wherein the N-alkyl-2-pyrrolidone is N-methylpyrrolidone.

26. The process according to claim 16, wherein three evaporator stages are provided.
